(19) United States
(12) Plant Patent Application Publication
(10) Pub. No.: US 2005/0010981 P1
(43) Pub. Date: Jan. 13, 2005

(54) Carmine strawberry plant

(50) FRAGARIA X ANANASSA; CARMINE

(76) Inventor: Craig K. Chandler, Dover, FL (US)

Correspondence Address: Thomas D. Stadsklev;Florida Foundation Seed Producers, Inc., P.O. Box 309, Greenwood, FL, 32443, US

(21) Appl. No.: 10/613,317

(22) Filed: Jul. 7, 2003

(51) Int. Cl. A01H 5/00

(52) U.S. Cl. PLT/208

(57) ABSTRACT

A new and distinct variety of strawberry (Fragaria×ananassa), which originated from seed produced by a hand-pollinated cross between ‘Rosa Linda’ and FL 93-53. The new strawberry, named ‘Carmine’, is distinguished by high December through February production of fruit that are firm, deep red, glossy and moderately resistant to Botrytis and anthracnose fruit rot diseases when grown in Dover, Fla. or other areas that have a subtropical climate similar to that of Dover.

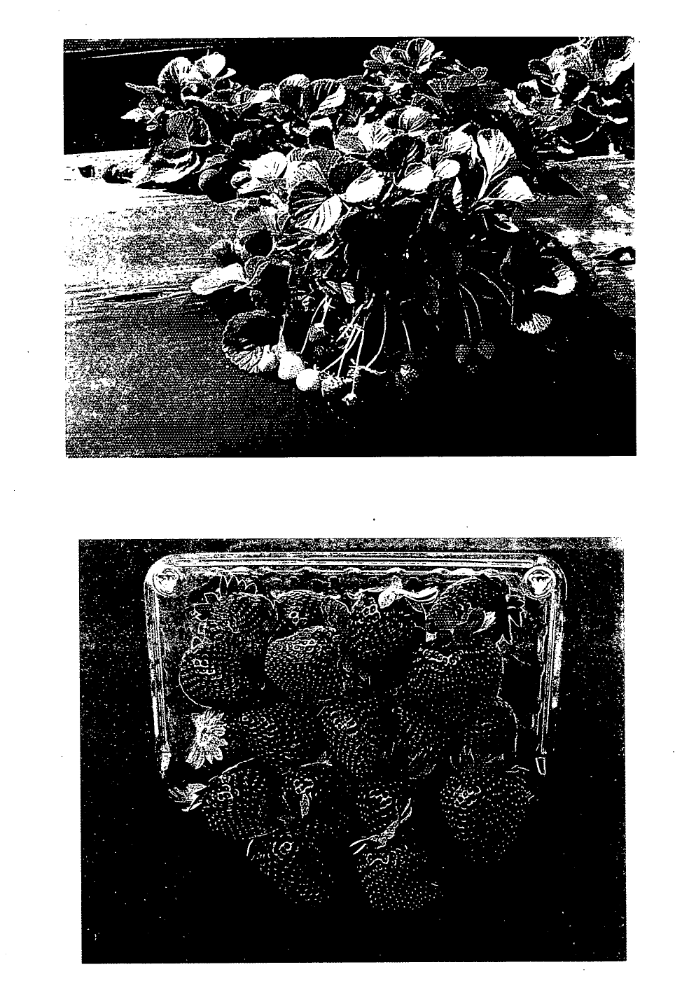

### BOTANICAL DESIGNATION

Fragaria×ananassa Duchesne

### BACKGROUND OF THE NEW VARIETY

The present invention relates to a new and distinct variety of strawberry (Fragaria×ananassa Duchesne) plant which is named ‘Carmine’ and more particularly to a strawberry plant that is distinguished by its high early season (December through February) production of firm, deep red fruit. Asexual propagation was performed at Dover, Fla. where the selection was made and plants were tested. Contrast is made to ‘Sweet Charlie’ (U.S. plant Pat. No. 8,729) and ‘Camarosa’ (U.S. plant Pat. No. 8,708), standard varieties, for reliable description. This new variety is a promising candidate for commercial success in that it has high early season fruit production like ‘Sweet Charlie’, but has significantly firmer fruit than ‘Sweet Charlie’.

### ORIGIN OF THE VARIETY

This strawberry plant (genotype) originated in a strawberry breeding plot at Dover, Fla. The seed parent was ‘Rosa Linda’ (U.S. plant Pat. No. 9,866), a strawberry variety with a desirable fruit shape and high early season yield potential. The pollen parent was FL 93-53 (not patented), a University of Florida breeding selection with the ability to produce firm attractive fruit. The seeds resulting from the controlled hybridization were germinated in a greenhouse and the resulting seedlings were planted and allowed to produce daughter plants by asexual propagation (i.e. by runners). Two daughter plants from each seedling were transplanted to raised beds, where they fruited. ‘Carmine’ strawberry (as represented by two daughter plants from the original seedling) exhibited attractive, firm fruit, and therefore was selected for further evaluation. ‘Carmine’ was selected from among 139 sibling genotypes as the 256th selection of the 1995-96 season, and thus was designated FL 95-256. It has been asexually propagated by runners, annually, and further test plantings have established that the vegetative and fruit characteristics of the propagules are identical to the initial two daughter plants.

### SUMMARY OF THE VARIETY

‘Carmine’, when grown in a subtropical fall and winter climate, is set apart from all other strawberry plants by a combination of the following characteristics: high December through February production (greater than 17,000 pounds of marketable fruit per acre); fruit that are firm, deep red, and glossy; and moderate resistance to Botrytis and anthracnose fruit rot diseases.

### BRIEF DESCRIPTION OF THE DRAWING

The accompanying photographs show a typical specimen of the plant and fruit as seen during the peak production of ‘Carmine’ in late February.

### DETAILED BOTANICAL DESCRIPTION

The following botanical description is that of mature plants of the variety grown under the ecological conditions (warm days, cool nights) prevailing at Dover, Fla. in late February. Colors are described using the Pantone® Color Formula Guide (www.pantone.com).

‘Carmine is a short day cultivar. It has a more compact plant habit than ‘Camarosa’ and ‘Rosa Linda’. Average height and width for mature plants is 19 cm and 26 cm respectively. Average petiole length and diameter is 15.6 cm and 2.5 mm respectively, and petioles have a medium pubescence. Average length and breadth of terminal leaflets is 60 and 59 mm respectively. Average length and breadth of secondary leaflets is 56 and 52 mm respectively. Leaflet margins are crenate and average 18 serrations per terminal leaflet, and 18 per secondary leaflet. The upper leaf surface is a dark grey green (Pantone®370 U); the lower leaf surface is a light grey green (Pantone®377 U); and the petiole is a medium yellow green (Pantone®397 U). Flowers open at or above the canopy, and have an average of 5 petals and 24 stamens. Individual petals have a length and width of 9 mm. The diameter of the corolla (i.e. the petals collectively) is 28 mm. The color of the calyx is yellow green (Pantone®363 U). Pedicels attached to mature primary fruit are 11 to 22 cm long, with branching of the inflorescence usually occurring very close to the crown. Mean fruit weight is less than or similar to that of ‘Sweet Charlie’ (Table 1 and 2) and ‘Rosa Linda’. Primary fruit are medium conic or wedge shaped (weighing 25-35 g); whereas secondary and tertiary fruit are mostly short conic (weighing 10-25 g). The external color of fully mature fruit is deep red (Pantone®1807C) and glossy; internal color is a warm red (Pantone® 1795C). The achenes are generally greenish yellow and level with or slightly protruding from the fruit surface. The calyx is generally medium in size and attractive. Fruit of ‘Carmine’ are firmer than those of ‘Sweet Charlie’ (Table 3) and ‘Rosa Linda’. The flavor of this fruit is acceptable, but not as highly regarded as that of ‘Camarosa’, ‘Sweet Charlie’, and ‘Strawberry Festival’ (U.S. plant Pat. No. 14,739) (Table 4). While generally sweet and juicy, the fruit, at times, can be slightly astringent. The preferred planting date for ‘Carmine’ is October 1 to October 15. Early season yields of ‘Carmine’ compared favorably to ‘Sweet Charlie’ and ‘Earlibrite’ (U.S. plant Pat. No. 13,061) during both the 1997-98 and 2001-02 seasons (Table 1 and 2). The December yield of ‘Carmine’ in 2001 was over twice that of ‘Sweet Charlie’. Yield during December can be important to a Florida grower's profitability. The average price per flat (10.25 lbs) during the five seasons between 1995 and 2000 was $17.38, $11.57, $10.51, and $7.27 for December, January, February, and March respectively (Florida Agricultural Statistics, www.nass.usda.pov/fl). ‘Carmine’ is moderately resistant to the two most serious disease problems on strawberry in Florida: Botrytis fruit rot (caused by Botrytis cinerea Pers.exFr.) and anthracnose fruit rot (caused by Colletotrichum acutatum Simmonds). In an unsprayed trial during the 2001-02 and 2002-03 seasons, 7.3 and 2.2% of the ‘Carmine’ fruit harvested from 19 February to 15 March showed symptoms of Botrytis fruit rot, compared to 18.7 and 6.8% for ‘Sweet Charlie’, the susceptible control. In another unsprayed trial during the 2001-02 and 2002-03 seasons, 9.6 and 9.3% of the ‘Carmine’ fruit harvested from 19 February to 22 March showed symptoms of anthracnose fruit rot, compared to 28.9 and 47.0% for ‘Strawberry Festival’, the susceptible control. The susceptibility of ‘Carmine’ to the twospotted spider mite (Tetranychus urtricae Koch) is unknown, but a serious infestation has not yet been observed in research center or commercial trials. DNA banding patterns for ‘Carmine’, ‘Sweet Charlie’, ‘Camarosa’, ‘Earlibrite’, ‘Oso Grande’ (U.S. plant Pat. No. 6,578) and ‘Strawberry Festival’ are presented in Table 5.

TABLE 1
Performance of strawberry cultivars at Dover, Fla. during the
1997-98 season^{z}.

Marketable yield (g/plant)

| | De- | | | | | Wt/fruit^{y} |
|---|---|---|---|---|---|---|
| Cultivar | cember | January | February | March | Total | (g) |
| Carmine | 114 a | 85 a | 246 ab | 212 b | 657 b | 16.4 c |
| Camarosa | 50 b | 105 a | 167 c | 426 a | 748 ab | 20.0 a |
| S. Charlie | 91 a | 54 b | 219 abc | 257 b | 622 b | 17.6 b |
| Earlibrite | 66 b | 110 a | 189 bc | 280 b | 645 b | 20.7 a |
| S. Festival | 58 b | 108 a | 255 a | 426 a | 847 a | 17.6 a |

^{z}Transplants were obtained from the following nursery locations: ‘Camarosa’ from a commercial nursery in North Carolina; all other entries were from GCREC-Dover. ‘Camarosa’ was planted on 16 Oct.; all other cultivars were planted on 9 Oct.
^{y}Mean fruit weight was determined by dividing total marketable fruit yield per plot by total marketable fruit number per plot.
^{x}Means based on four replications. Mean separation within columns by Fisher's protected LSD test, P ≦ 0.05.

TABLE 2
Performance of strawberry cultivars at Dover, Fla. during
the 2001-02 season^{z}.

| | Marketable yield (g/plant) | | | | |
|---|---|---|---|---|---|
| Cultivar | December | January | February | Total | Wt/fruit^{y} (g) |
| Carmine | 232 a | 62 b | 204 a | 499 a | 17.0 b |
| S. Charlie | 102 c | 92 a | 166 a | 360 b | 16.4 b |
| Earlibrite | 205 ab | 33 c | 201 a | 439 a | 21.1 a |
| S. Festival | 163 b | 61 b | 221 a | 444 a | 17.2 b |

^{z}All transplants were obtained from a commercial nursery in North Carolina, and planted on 9 Oct.
^{y}Mean fruit weight was determined by dividing total marketable fruit yield per plot by total marketable fruit number per plot.
^{x}Means based on four replications. Mean separation within columns by Fisher's protected LSD test, P ≦ 0.05.

TABLE 3
Physical and chemical characteristics of strawberry fruit harvested at
Dover, Fla. Feb. 23, 1998 and Feb. 25, 2002^{z}.

| | |
|---|---|
| | Firmness |
| L value^{y} | (kg force) |

| Cultivar | 1998 | 2002 | 1998 | 2002 |
|---|---|---|---|---|
| Carmine | 30.5 c | 35.7 b | 0.38 a | 0.41 a |
| Camarosa | 29.6 c | 34.2 b | 0.43 a | 0.27 b |
| Sweet Charlie | 35.1 a | 38.2 a | 0.22 b | 0.30 b |
| S. Festival | 32.6 b | 35.6 b | 0.38 a | 0.42 a |

| | |
|---|---|
| Soluble | Titratable acidity |
| solids (%) | (% citric acid) |

| Cultivar | 1998 | 2002 | 1998 | 2002 |
|---|---|---|---|---|
| Carmine | 8.6 | 6.5 | 0.77 | 0.86 |
| Camarosa | 7.7 | 8.5 | 0.87 | 0.93 |
| Sweet Charlie | 8.0 | 7.3 | 0.65 | 0.75 |
| S. Festival | 7.7 | 8.0 | 0.77 | 0.84 |

^{z}L and firmness values are the average of eight observations. Mean separation within columns by Duncan's multiple range test, P ≦ 0.05.
^{y}The lower the value, the darker the color.

TABLE 4
Sensory characteristics of strawberry fruit harvested at
Dover, Fla. Feb. 25, 2002^{z}.

| Cultivar | Appearance | Firmness | Flavor | Sweetness |
|---|---|---|---|---|
| Carmine | 7.2 a | 7.1 b | 5.3 c | 4.8 c |
| Camarosa | 6.7 b | 7.0 bc | 6.8 a | 6.6 a |
| Sweet Charlie | 6.2 b | 6.6 c | 6.2 b | 6.0 b |
| S. Festival | 7.5 a | 7.5 a | 7.2 a | 6.8 a |

^{z}Means based on the ratings of 72 untrained panelists. Mean separation within columns by Duncan's multiple range test, P ≦ 0.05. Characteristics are rated on a 1-9 hedonic scale, with 1 = dislike extremely, 5 = neither like nor dislike, and 9 = like extremely.

TABLE 5
DNA marker analysis^{z}

| | Primer | | | | |
|---|---|---|---|---|---|
| | B06 | B07 | B14 | X11 | X06 |
| | Band number for each primer and DNA pattern | | | | |
| Variety | 123 | 1234 | 12345 | 1 | 12 |
| Carmine | 000 | 1101 | 00000 | 0 | 10 |
| Earlibrite | 101 | 1101 | 00000 | 0 | 11 |
| Sweet Charlie | 011 | 0101 | 00111 | 1 | 01 |
| Camarosa | 101 | 1010 | 00010 | 1 | 11 |
| Oso Grande | 001 | 1011 | 11010 | 1 | 10 |
| S. Festival | 000 | 1001 | 00010 | 1 | 10 |
| ^{z}Random amplified polymorphic DNA (RAPD) patterns were determined using primers B06, B07, B14, X06, and X11 from Operon Technologies, Inc. Stolon tip DNA's were isolated using DNeasy Plant ™ extraction kit from Qiagen ®, Inc.. Amplification reactions were performed in | | | | | |
| # 20 microliter volumes using a procedure adapted from Williams et al., 1990), Nucleic Acids Research 25: 6531-6535. The reagents and conditions included 50 mM Tris (pH 8.3), 0.25 mg/mL bovine serum albumin, 2. | | | | | |
| # dGTP, dTTP, 1.0 mM primer DNA, 0.065 ng strawberry DNA, 1 unit Taq-DNA polymerase (Promega, Inc.). The reaction conditions were 4 minutes at 94° C., then 10 seconds at 94° C., 1 minute at 45° C., 3.5 minutes at 68° C., then 9 cycles of 10 seconds at 94° C., 1 minute at | | | | | |
| # 45° C. with an incrementation of 0.5 degrees per cycle, 3.5 minutes at 68° C., then 29 cycles of 10 seconds at 94° C., 1 minute at 40° C. and 3.5 minutes at 68° C. with a 10 second extens | | | | | |
| # The reaction products were analyzed with gel electrophoresis using 1.0% agarose 3:1 high resolution blend (AMRESCO, Inc.) in a running buffer of 0.045 M Tris-Borate, 0.001 M EDTA. The separated DNA was detected using ethidium bromide and viewed with a ultra violet transilluminator. Reproducible | | | | | |
| # polymorphic banding from the electrophoresis analysis was observed with the DNA primers. The amplification reactions resulted with varying levels of polymorphism, from 2 to 5 polymorphic bands depending on the primer used. The polymorphic bands were scored as 0 equals absence and 1 equals presence. | | | | | |

1. A new and distinct strawberry plant as illustrated and described, characterized by high December through February production of fruit that are firm, deep red, glossy and moderately resistant to Botrytis and anthracnose fruit rot diseases when grown in the Dover/Plant City area of Florida.
